(19) United States
(12) Patent Application Publication
(10) Pub. No.: US 2026/0315166 A1
(43) Pub. Date: Oct. 15, 2026

(54) A SYSTEM AND METHOD FOR MIXED REALITY TELE PROCTORING IN MINIMALLY INVASIVE SURGERY

(71) Applicant: SSI IP HOLDINGS INC., Ft. Lauderdale, FL (US)

(72) Inventors: Sudhir Prem Srivastava, Haryana (IN); Vishwajyoti Pascual Srivastava, Haryana (IN); S Naveen Ajay Kumar, Haryana (IN); Aviral Dwivedi, Haryana (IN); Anuj Saini, Haryana (IN); Ravinder Singh Rautela, Haryana (IN); S Rohan Akshai Kumar, Haryana (IN)

(21) Appl. No.: 19/106,774

(22) PCT Filed: Jul. 15, 2024
(86) PCT No.: PCT/IN2024/051242

§ 371 Date: Feb. 26, 2025

(30) Foreign Application Priority Data
Nov. 10, 2023 (IN) 202311076797

(51) Int. Cl. A61B 34/35; A61B 1/00; A61B 17/00; A61B 34/00; A61B 34/30; A61B 90/00

(52) U.S. Cl. CPC A61B 34/35; A61B 1/00193; A61B 34/25; A61B 90/36; A61B 2017/00207; A61B 2034/305; A61B 2090/365

(57) ABSTRACT

The inventions relate to a mixed reality tele proctoring system for use in surgical procedures, comprising a surgeon module, a network architecture based on WebRTC for low latency communication, and a proctor module, wherein the system enables real-time audiovisual communication and annotation between a surgeon and a proctor. The surgeon module is adapted to process and transmit 2D and 3D stereoscopic imagery from a wide range of endoscopic cameras. The network architecture employs a signaling server for handshake, exchanging encoding formats, and bitrate information and creates a peer-to-peer encrypted communication link between the proctor and the surgeon. The proctor module allows adjustments for screen size and inter-pupillary distance and allows proctor to provide suggestions via voice and annotations on a virtual annotation panel. Multiple proctors could connect to the same operation theatres which eases methods of live surgery streaming for educational purposes.

### CROSS REFERENCE TO RELATED APPLICATIONS

This application is a U.S. National Stage under 35 U.S.C. §371 of International Application No. PCT/IN2024/051242 filed on Jul. 15, 2024, which claims priority to Indian Patent Application No. 202311076797 filed on Nov. 10, 2023, and entitled “A SYSTEM AND METHOD FOR MIXED REALITY TELE PROCTORING IN MINIMALLY INVASIVE SURGERY”, which are incorporated herein by reference in its entirety.

It is intended that the referenced application may be applicable to the concepts and embodiments disclosed herein, even if such concepts and embodiments are disclosed in the referenced applications with different limitations and configurations and described using different examples and terminology.

### TECHNICAL FIELD

The present disclosure generally relates to the field of robotic surgical system for minimally invasive surgery, and more particularly, the disclosure relates to a mixed reality tele proctoring module to be used in a multi-arm robotic surgery environment in medical applications.

### BACKGROUND

This section is intended to introduce the reader to various aspects of art that may be related to various aspects of the present disclosure, which are described below. This disclosure is believed to be helpful in providing the reader with background information to facilitate a better understanding of the various aspects of the present disclosure. Accordingly, it should be understood that these statements are to be read in this light, and not just as an admissions of prior art.

Robotic assisted surgical systems have been adopted worldwide to gradually replace conventional surgical procedures such as open surgery and laparoscopic surgical procedures. The robotic assisted surgery offers various benefits to a patient during surgery and during post-surgery recovery time. The robotic assisted surgery equally offers numerous benefits to a surgeon in terms of enhancing the surgeon's ability to precisely perform surgery, less fatigue and a magnified clear three-dimensional (3D) vision of a surgical site. Further, in a robotic assisted surgery, the surgeon typically operates with a hand controller/master controller/surgeon input device/joystick at a surgeon console system to seamlessly receive and transfer complex actions performed by him/her giving the perception that he/she himself/herself is directly articulating a surgical tools/surgical instrument to perform the surgery. The surgeon operating on the surgeon console system may be located at a distance from a surgical site or may be located within an operating theatre where the patient is being operated on. The robotic assisted surgical systems may comprise of multiple robotic arms aiding in conducting robotic assisted surgeries.

The robotic assisted surgical system utilizes a sterile adapter/a sterile barrier to separate a non-sterile section of the multiple robotic arms from a mandatory sterile surgical tools/surgical instrument attached to one end of the multiple robotic arms. The sterile adaptor/sterile barrier may include a sterile plastic drape that envelops the multiple robotic arms and the sterile adaptor/sterile barrier that operably engages with the sterile surgical tools/surgical instrument in the sterile field.

One of the main challenges is the unavailability of real-time guidance and supervision from experienced proctors to medical professionals/surgeons, while performing robotic surgical procedures. Another challenge is that the existing methods for tele-proctoring methods have limitations like higher latency, complexity, and lack of immersive interaction. Further, another challenge is that the proctors often don't receive a real-time 3D reconstruction of the endoscopic view of the surgical site, leading to wrong judgement.

In the light of the above-mentioned challenges, there is a need for providing an improved tele proctoring method for surgeons in a multi-arm robotic surgical system which will solve the above-mentioned problems related to robotic assisted surgeries.

### SUMMARY OF THE DISCLOSURE

Some or all of the above-mentioned problems related to providing training to the surgeons and OT staff are proposed to be addressed by certain embodiments of the present disclosure.

According to an aspect of the invention, there is disclosed a A mixed reality tele-proctoring system in a multi-arm robotic surgical system comprising an operating table around which one or more robotic arms are arranged and a surgeon console, the mixed reality tele-proctoring system comprising: a surgeon module to be used by a surgeon, the surgeon module comprising: a processor coupled to an encryptor, the processor configured to receive a video stream from an endoscopic camera; the encryptor configured to encrypt the received video stream; an input device; an output device; and a two-dimensional (2D) touch screen monitor coupled to the processor, the 2D touch screen monitor configured to be used as a graphical user interface to capture inputs from the surgeon; a proctor module to be used by a proctor comprises: a head mounted device including a processor operably coupled to a webRTC architecture, a 3D stereoscopic display, and a speaker, the processor configured to process an endoscope feed received through the webRTC architecture and render as a stereoscopic 3D image for left and right eyes on the 3D stereoscopic display, and a virtual annotation panel to add annotations by the proctor, and the webRTC architecture configured to exchange signals between the surgeon module and the proctor module.

According to an embodiment of the invention, the input device may be anyone out of a microphone, or a web enabled input device to input a 3D video stream, like a camera.

According to another embodiment of the invention, the virtual annotation panel is configured to capture hand gestures of the proctor.

According to yet another embodiment of the invention, the proctor can provide annotations and voice instructions to the surgeon.

According to yet another embodiment of the invention, the proctor can control the screen size adjustments and inter-pupillary distance (IPD) adjustments which provide custom controls and adjustments.

According to yet another embodiment of the invention, the proctor module supports ultra-low latency audio communication and advanced mixed reality headsets for annotation and synchronization with the console used by the surgeon.

According to still another embodiment of the invention, the proctor can choose to connect with a correct operation theatre from the list of all live cases being proctored.

According to still another embodiment of the invention, the proctor module allows adjustments for screen size and inter-pupillary distance.

According to still another embodiment of the invention, the webRTC architecture creates a peer-to-peer connection between the surgeon module and the proctor module.

According to still another embodiment of the invention, the creation of the peer-to-peer connection between the surgeon module and the proctor module comprises: gathering of network addresses of the surgeon module and the proctor module through STUN (Session Traversal Utilities for NAT) servers; exchanging the gathered addresses; and establishing a peer-to-peer encrypted direct connection between the surgeon module and the proctor module.

According to still another embodiment of the invention, the webRTC architecture utilizes a signaling server to establish a direct connection between the surgeon and the proctor.

According to still another embodiment of the invention, multiple proctors can connect to the feed from an operation theatre, received through the webRTC architecture.

According to still another embodiment of the invention, a 360° camera can be installed in the operation theatre to provide a real time operation theatre view.

According to still another embodiment of the invention, the proctor can supervise the real time operation theatre view.

Other embodiments, systems, methods, apparatus aspects, and features of the invention will become apparent to those skilled in the art from the following detailed description, the accompanying drawings, and the appended claims.

### BRIEF DESCRIPTION OF THE DRAWINGS

The summary above, as well as the following detailed description of the disclosure, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to the scale. Wherever possible, like elements have been indicated by identical numbers.

Embodiments of the present disclosure will now be described, by way of example only, with reference to the following diagrams wherein:

FIG. 1 illustrates an example implementation of a multi-arm teleoperated surgical system which can be used with one or more features in accordance with an embodiment of the disclosure;

FIG. 2 illustrates a surgeon side module in accordance with an embodiment of the disclosure;

FIG. 3 illustrates a proctor side module in accordance with an embodiment of the disclosure;

FIG. 4 illustrates a webRTC architecture in accordance with an embodiment of the disclosure; and

FIG. 5 illustrates a tele proctoring module in accordance with an embodiment of the disclosure.

### DETAILED DESCRIPTION OF THE DISCLOSURE

For the purpose of promoting an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof. Throughout the patent specification, a convention employed is that in the appended drawings, like numerals denote like components.

Reference throughout this specification to “an embodiment”, “another embodiment”, “an implementation”, “another implementation” or similar language means that a particular feature, structure, or characteristic described in connection with the embodiment is included in at least one embodiment of the present disclosure. Thus, appearances of the phrase “in an embodiment”, “in another embodiment”, “in one implementation”, “in another implementation”, and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures proceeded by “comprises... a” does not, without more constraints, preclude the existence of other devices or other sub-systems or other elements or other structures or additional devices or additional sub-systems or additional elements or additional structures.

Unless otherwise defined, all technical and scientific terms used herein have the same meaning as commonly understood by one of ordinary skill in the art to which this disclosure belongs. The device, system, and examples provided herein are illustrative only and not intended to be limiting.

The terms “a” and “an” herein do not denote a limitation of quantity, but rather denote the presence of at least one of the referenced items. Further, the term sterile barrier and sterile adapter denotes the same meaning and may be used interchangeably throughout the description.

Embodiments of the disclosure will be described below in detail with reference to the accompanying drawings.

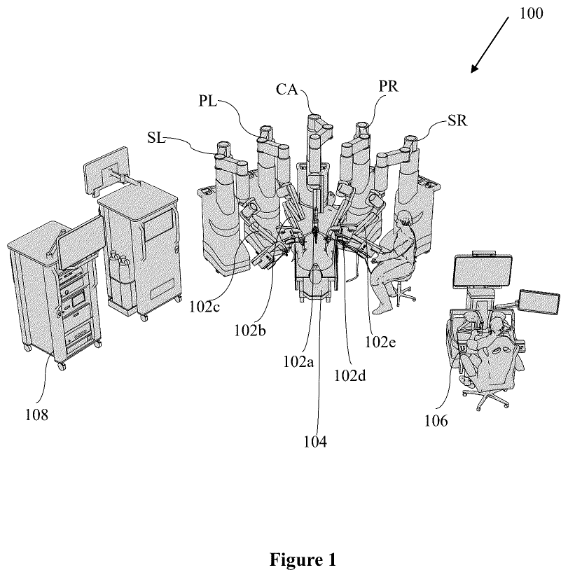

FIG. 1 illustrates an example implementation of a multi arm teleoperated surgical system which can be used with one or more features in accordance with an embodiment of the disclosure. Specifically, FIG. 1 illustrates the multi arm teleoperated surgical system (100) having five robotic arms (102a), (102b), (102c), (102d), (102d), (102e), mounted on five robotic arm carts around an operating table (104). The five-robotic arms (102a), (102b), (102c), (102d), (102e), as depicted in FIG. 1, are for illustration purposes and the number of robotic arms may vary depending upon the type of surgery. The exemplary five robotic arms (102a), (102b), (102c), (102d), (102e), are arranged along the operating table (104) and may be arranged in different manner but not limited to the robotic arms (102a), (102b), (102c), (102d), (102e), arranged along the operating table (104). The robotic arms (102a), (102b), (102c), (102d), (102e), may be separately mounted on the five robotic arm carts or the robotic arms (102a), (102b), (102c), (102d), (102e), mechanically and/or operationally connected with each other or the robotic arms (102a), (102b), (102c), (102d), (102e), connected to a central body (not shown) such that the robotic arms (102a), (102b), (102c), (102d), (102e), branch out of a central body (not shown). Further, the multi arm teleoperated surgical system (100) may include a surgeon console (106), a vision cart (108), and a surgical instrument and accessory table.

In surgical procedures, particularly in environments such as operating theatres (OT), it is essential to provide real-time guidance and supervision to medical professionals, especially surgeons, from experienced proctors. Traditional methods for tele-proctoring have limitations, including latency, complexity, and lack of immersive interaction, proctors often don't receive a 3D reconstruction of the endoscopic view which can lead to a misjudgment.

While remote monitoring and proctoring have the potential to transform the medical field, it is imperative to recognize the intricacies involved. Specialized skills in telecommunications, data security, technical adaptability, and network engineering are pivotal for overcoming the challenges and ensuring the successful operation of such systems. As the demand for remote surgical guidance grows, so does the need for experts who can navigate the complexities of this innovative technology. Their contributions are critical in advancing patient care and medical practices.

The mixed reality tele proctoring module for use in surgical procedures, includes a Surgeon Side Module, a Network Architecture based on WebRTC for low latency communication, and a Proctor Side Mixed Reality Headset, wherein the system enables real-time audiovisual communication and annotation between surgeons and proctors.

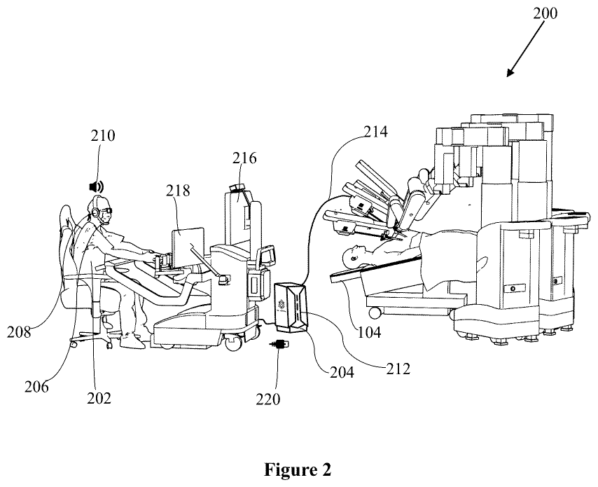

FIG. 2 illustrates a surgeon module in accordance with an embodiment of the disclosure. This surgeon module (200) serves as the interface for a surgeon (202) within the operating theatre. The surgeon module (200) comprises of a processor (204), an input device (206), an output device (208, 210), an encryptor (212), and an endoscopic camera (214). The input device (206) may be anyone out of a microphone (206), or a web enabled input device to input a 3D video stream, like a camera (214). The surgeon module (200) is capable of receiving endoscopic signals from the endoscopic camera (214), including 3D stereoscopic imagery display (216) and 2D display (218) for mono endoscope feeds along with 360 operation theatre capture is an unparalleled advantage. The endoscopic camera (214) continuously sends a video stream to the processor (204). The output device may be a speaker (210), or a 2D display (218), etc. The processor (204) has the encryptor (212) which is configured to encrypt the received video stream and sends the encrypted video to the 2D display (218). The surgeon module (200) is adapted to process and transmit 2D and 3D stereoscopic imagery from a wide range of the endoscopic camera (214).

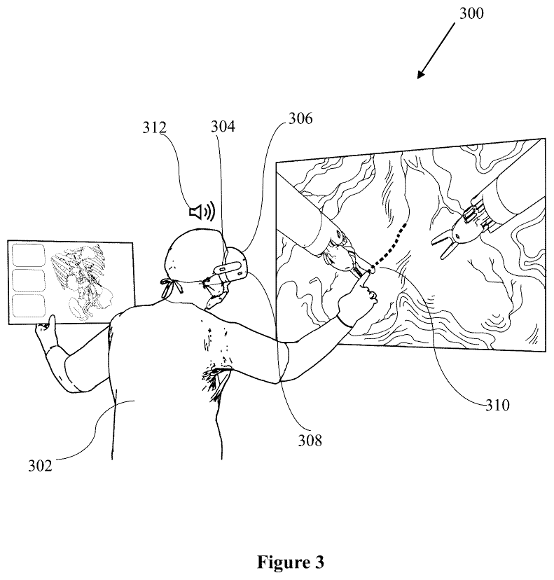

FIG. 3 illustrates a proctor module in accordance with an embodiment of the disclosure. The proctor module (300) is designed to be used by a proctor (302). The proctor module (300) comprises of a head mounted device (304) including a processor (306), a 3D stereoscopic display (308), and a virtual annotation panel (310) to capture hand gestures of the proctor (302). The proctor module (300) processes and renders the received endoscope feed as a stereoscopic 3D image for left and right eyes on the 3D stereoscopic display (308). The head mounted device (304) is a headset which creates the stereoscopic depth vision for the proctor (302). The proctor (302) wears the head mounted device (304) and sees the 3D video stream on the 3D stereoscopic display (308) of the head mounted device (304). The proctor (302) can add annotations by simply writing anything virtually using hand gestures. The annotations and voice instructions by an experienced surgeon/proctor can be provided to the surgeon (202) performing surgery in the operation theatre for better surgery outcome. The proctor (302) can control the screen size adjustments and inter-pupillary distance (IPD) adjustments which provide custom controls and adjustments. The proctor module (302) supports ultra-low latency audio communication and advanced mixed reality headsets (304) for annotation and synchronization with the console used by the surgeon (202). The proctor (302) can choose to connect with correct operation theatre from the list of all live cases at the moment. The proctor module (300) allows adjustments for screen size and inter-pupillary distance. Further, the proctor module (300) allows the proctor (302) to provide suggestions via voice using a speaker (312) and annotations.

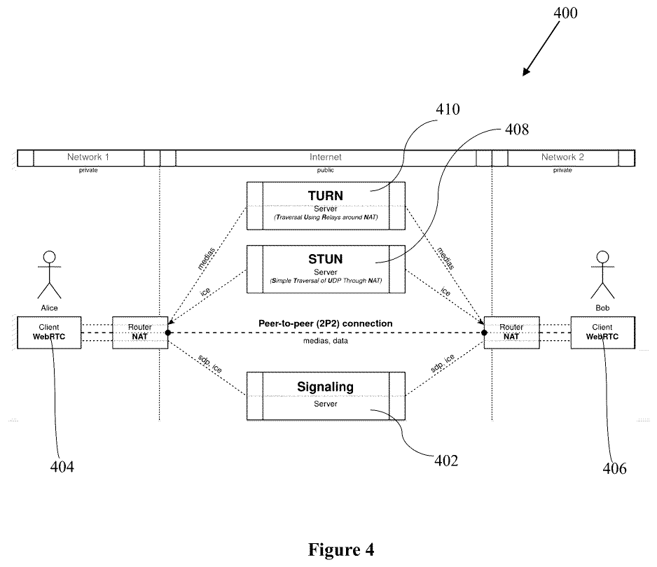

FIG. 4 illustrates the webRTC architecture (400) for exchange of signals between the surgeon module (200) and the proctor module (300). Creating a peer-to-peer connection in WebRTC involves several steps, including gathering network addresses through STUN (Session Traversal Utilities for NAT) servers, signaling to exchange these addresses, and ultimately establishing a direct connection between peers.

Here's an overview of this process: 1. Peer Initialization: a. Both the sender (Peer A) and receiver (Peer B) initialize their WebRTC applications, setting up the necessary components for real-time communication. 2. Network Address Gathering (STUN): a. Peer A and Peer B use the STUN server to gather their network addresses. STUN helps discover the public IP address and port through which each peer can be reached on the internet. b. For example, Peer A sends a request to the STUN server, which replies with the public IP address and port number that Peer A is using to communicate with the STUN server. Similarly, Peer B does the same. 3. Signaling: a. Peer A and Peer B exchange information about their network addresses using a signaling server. Signaling doesn't transmit media but is responsible for coordinating the negotiation between the peers. b. Peer A generates an “offer” message that includes its network address information (gathered in Step 2) and the types of media and codecs it can handle. This offer is sent to the signaling server. c. The signaling server forwards Peer A's offer to Peer B. d. Peer B receives the offer from the signaling server. It also generates an “answer” message in response. The answer includes Peer B's network address information and its preferred media settings. e. The answer is sent back to the signaling server, which in turn forwards it to Peer A.

4. ICE (Interactive Connectivity Establishment): a. Before establishing a direct connection, both Peer A and Peer B use the ICE framework to determine the best possible network path to reach each other. b. ICE gathers a list of network candidates, which may include public IP addresses and ports, local IP addresses, and relay candidates (in case direct P2P connections are not possible due to NAT or firewall restrictions). c. The local candidates are used for communication within the same local network, and the public candidates are used to communicating over the internet. 5. Direct Peer-to-Peer Connection: a. With the network address information obtained through STUN and the negotiated settings from the signaling process, both Peer A and Peer B attempt to connect directly to each other. b. They use the network addresses (public IP and port) provided during the ICE process to initiate a direct connection. This connection can traverse NATs and firewalls, as ICE selects the most appropriate network path. c. Once the direct connection is successfully established, Peer A and Peer B can exchange media and data directly without the need for relay servers. The combination of STUN for network address retrieval, signaling for offer/answer exchange, and ICE for network path discovery allows WebRTC to set up secure and efficient peer-to-peer connections, enabling real-time communication between users over the internet.

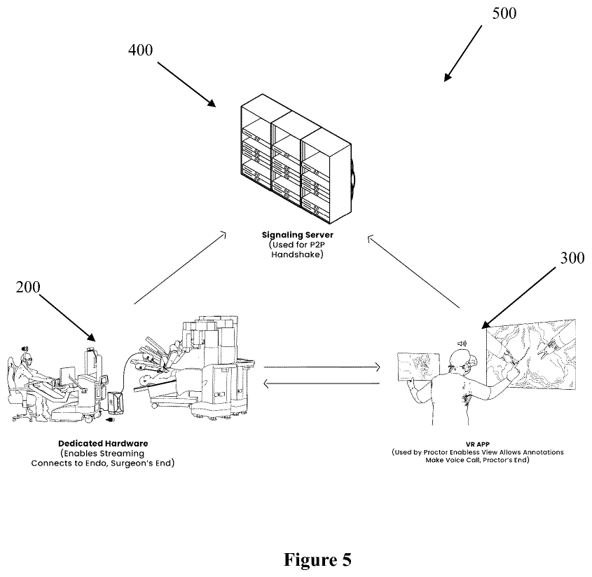

FIG. 5 illustrates a system for mixed reality tele proctoring module in minimally invasive surgery in accordance with an embodiment of the disclosure. The mixed reality tele proctoring module (500) serves as the interface for the surgeon (202) within the operation theatre. The mixed reality tele proctoring module (500) is capable of receiving endoscopic signals, including 3D stereoscopic imagery and 2D mono endoscope feeds along with 360° capture of the operation theatre is an unparalleled advantage. This mixed reality tele proctoring module (500) connects to a signaling server (402) and streams the video feed. It manages 2-way audio transmission and annotation reception which allows a clear communication between the surgeon (202) and the proctor (302).

The invention utilizes a WebRTC-based, ultra-low latency peer-to-peer architecture (400) as shown in FIG. 4, the role of a central server (402) is minimum which reduces latency and network overhead. A server (402), acting as a signaling server (e.g., Amazon Azure Virtual Desktop), facilitates the handshake between the surgeon (202) and the proctor (302). The server (402) exchanges critical information of the two nodes (404, 406) for communication, such as IP addresses encoder formats and bitrate etc. This information is referred to as ICE Candidates. The STUN (408)/TURN (410) server takes the charge after the initial handshake and handles a seamless peer to peer communication. The architecture (400) employs a signaling server (402) for handshake, exchanging encoding formats, and bitrate information and creates a peer-to-peer encrypted communication link between the proctor (302) and the surgeon (202). Multiple proctors could connect to the same operation theatre which eases methods of live surgery streaming for educational purposes.

The present disclosure has the following advantages: The surgeon module supports a very wide range of endoscopic devices and cameras. It allows usage of mono and stereo endoscopic views and supports line by line and side by side stereo formats. The utilization of an ultra-low latency architecture (WebRTC) (400) ensures real-time communication and feedback. The proctor (302) can provide guidance using a mixed reality headset, enhancing the quality of support. The proctor (302) can annotate the findings and communicate over voice. The proctor (302) can also supervise the real time operation theatre view using a 360-camera feed. The proctor (302) can view the 3D endoscopic feed on a monitor instead of a headset. There can be multiple proctors or viewers, who can subscribe to the feed from an operation theatre. This allows multiple supervisors to review at once. The system enables real-time audiovisual communication and annotation between surgeons and proctors.

The foregoing descriptions of exemplary embodiments of the present disclosure have been presented for purposes of illustration and description. They are not intended to be exhaustive or to limit the disclosure to the precise forms disclosed, and obviously many modifications and variations are possible in light of the above teaching. The exemplary embodiment was chosen and described in order to best explain the principles of the disclosure and its practical application, to thereby enable others skilled in the art to best utilize the disclosure and various embodiments with various modifications as are suited to the particular use contemplated. It is understood that various omissions, substitutions of equivalents are contemplated as circumstance may suggest or render expedient but is intended to cover the application or implementation without departing from the spirit or scope of the claims of the present disclosure.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

While specific language has been used to describe the disclosure, any limitations arising on account of the same are not intended. As would be apparent to a person in the art, various working modifications may be made to the apparatus in order to implement the inventive concept as taught herein.

We claim:

1. A mixed reality tele-proctoring system in a multi-arm robotic surgical system comprising an operating table around which one or more robotic arms are arranged and a surgeon console, the mixed reality tele-proctoring system comprising:
a surgeon module to be used by a surgeon, the surgeon module comprising:
a processor coupled to an encryptor, the processor configured to receive a video stream from an endoscopic camera;
the encryptor configured to encrypt the received video stream;
an input device;
an output device; and
a two-dimensional (2D) touch screen monitor coupled to the processor, the 2D touch screen monitor configured to be used as a graphical user interface to capture inputs from the surgeon;
a proctor module to be used by a proctor comprises:
a head mounted device including a processor operably coupled to a webRTC architecture, a 3D stereoscopic display, and a speaker, the processor configured to process an endoscope feed received through the webRTC architecture and render as a stereoscopic 3D image for left and right eyes on the 3D stereoscopic display, and
a virtual annotation panel to add annotations by the proctor, and
the webRTC architecture configured to exchange signals between the surgeon module and the proctor module.

2. The mixed reality tele-proctoring system as claimed in claim 1, wherein the input device may be anyone out of a microphone, or a web enabled input device to input a 3D video stream, like a camera.

3. The mixed reality tele-proctoring system as claimed in claim 1, wherein the virtual annotation panel is configured to capture hand gestures of the proctor.

4. The mixed reality tele-proctoring system as claimed in claim 1, wherein the proctor can provide annotations and voice instructions to the surgeon.

5. The mixed reality tele-proctoring system as claimed in claim 1, wherein the proctor can control the screen size adjustments and inter-pupillary distance (IPD) adjustments which provide custom controls and adjustments.

6. The mixed reality tele-proctoring system as claimed in claim 1, wherein the proctor module supports ultra-low latency audio communication and advanced mixed reality headsets for annotation and synchronization with the console used by the surgeon.

7. The mixed reality tele-proctoring system as claimed in claim 1, wherein the proctor can choose to connect with a correct operation theatre from the list of all live cases being proctored.

8. The mixed reality tele-proctoring system as claimed in claim 1, wherein the proctor module allows adjustments for screen size and inter-pupillary distance.

9. The mixed reality tele-proctoring system as claimed in claim 1, wherein the webRTC architecture creates a peer-to-peer connection between the surgeon module and the proctor module.

10. The mixed reality tele-proctoring system as claimed in claim 9, wherein the creation of the peer-to-peer connection between the surgeon module and the proctor module comprises:
gathering of network addresses of the surgeon module and the proctor module through STUN (Session Traversal Utilities for NAT) servers;
exchanging the gathered addresses; and
establishing a peer-to-peer encrypted direct connection between the surgeon module and the proctor module.

11. The mixed reality tele-proctoring system as claimed in claim 9, wherein the webRTC architecture utilizes a signalling server to facilitate handshake between the surgeon and the proctor.

12. The mixed reality tele-proctoring system as claimed in claim 1, wherein multiple proctors can connect to the feed from an operation theatre, received through the webRTC architecture.

13. The mixed reality tele-proctoring system as claimed in claim 1, wherein a 360° camera can be installed in the operation theatre to provide a real time operation theatre view.

14. The mixed reality tele-proctoring system as claimed in claim 13, wherein the proctor can supervise the real time operation theatre view.
